(19) United States
(12) Patent Application Publication
(10) Pub. No.: US 2024/0252234 A1
(43) Pub. Date: Aug. 1, 2024

(54) SYSTEM AND METHOD FOR DETERMINING ABLATION LESION STATE

(71) Applicant: KONINKLIJKE PHILIPS N.V., EINDHOVEN (NL)

(72) Inventors: Alon RAZON, Caesarea (IL); Yotam REISNER, Caesarea (IL); Yael PISTONOVICH, Caesarea (IL); Alisa KOMLEVA, Caesarea (IL); Asi ELAD, Caesarea (IL); Gal SHLEIFER, Caesarea (IL); Yehonatan BEN DAVID, Caesarea (IL); Zalman IBRAGIMOV, Caesarea (IL)

(21) Appl. No.: 17/246,768

(22) Filed: May 3, 2021

Related U.S. Application Data
(60) Provisional application No. 63/018,715, filed on May 1, 2020.

(51) Int. Cl. A61B 18/14; A61B 18/12; G06N 20/00

(52) U.S. Cl. CPC A61B 18/1492; A61B 18/1206; G06N 20/00; A61B 2018/0072; A61B 2018/0075; A61B 2018/00755; A61B 2018/00761; A61B 2018/00767; A61B 2018/1467; A61B 2560/0487

(57) ABSTRACT

A controller, system and method for determining whether an ablation performed by an ablation catheter creates a lesion having a depth reaching or exceeding a predetermined depth, e.g. whether the lesion is transmural. The system comprises an ablation catheter with at least two electrodes for performing an ablation on tissue. One of the electrodes is an ablation electrode and the other electrode or electrodes are reference electrodes. A processor is used to determine, e.g. in real time, whether the depth of the lesion resulting from the ablation has reached or exceeded the predetermined depth based on ablation-dependent variables. The ablation-dependent variables may comprise an electrical signal received by the ablation electrode and one or more impedance values between the electrodes. A machine learning algorithm can thus be used to classify the tissue which is being ablated. The inputs, i.e. the input data, of the machine learning algorithm are features derived from the ablation-dependent variables, wherein the features correlate with the depth of a lesion resulting from the ablation. The machine learning algorithm is adapted to output, in real time, a classification comprising an indication whether or not a depth of the lesion resulting from the ablation reaches or exceeds a predetermined depth.

### FIELD OF THE INVENTION

This invention relates to the field of tissue ablation. In particular, it relates to systems, methods, computer programs and media comprising such programs for determining whether an ablated lesion has reached or exceeded a predetermined depth for example for determining whether a lesion is transmural or not.

### BACKGROUND OF THE INVENTION

Cardiac ablation is a procedure used to remove or terminate a faulty electrical activation pathway from sections of the hearts of those who are prone to developing cardiac arrhythmias. To do so, heart tissue is burned (by heat or cold) such that a scar tissue is produced, thereby “electrically isolating” a non-healthy region from a healthy one.

A successful lesion needs to incorporate the targeted tissue completely and result in irreversible cellular injury and death of the entire thickness of the tissue (i.e. transmurality) to block impulse conduction while leaving the surrounding structures unaffected.

To maximize efficacy and safety, lesion formation has traditionally been assessed with surrogate measures including radio-frequency power, temperature, impedance change, change in electrogram amplitude, contact force, duration of energy delivery, and different indices that attempt to combine surrogate measures, e.g. Ablation Index (AI) and Lesion Index (LSI). Although very useful, these parameters are imprecise and do not inform on whether a particular lesion depth has been attained, e.g. in other words whether a lesion is to be considered transmural or not. Direct, real time imaging of the lesion formation would enable better control of lesion formation to improve efficacy and safety.

### SUMMARY OF THE INVENTION

The invention is defined by the claims.

According to examples in accordance with an aspect of the disclosure, there is provided a processing system for determining whether an ablation of tissue (108) of a subject performed using an ablation catheter (102) creates a lesion having a depth that reaches or exceeds a predetermined depth, wherein the ablation catheter (102) comprises at least two electrodes, wherein at least one of the electrodes is an ablation electrode (104) and the other electrode or electrodes are reference electrodes (106), wherein, during the ablation, electrical signals are measured at the at least two electrodes which electrical signals comprise one or more ablation dependent variables, the processing system comprising:
an input configured to receive data comprising the one or more ablation dependent variables;
a processor (202) configured to process the received data and:
   derive one or more features (210) from the one or more ablation dependent variables, the one or more features (210) correlating with depth of the lesion resulting from the ablation;
   generate an ablation classification result using a machine learning algorithm (212) trained to provide the classification result by processing input data, the classification result comprising an indication on whether or not a depth of the lesion resulting from the ablation reaches or exceeds a predetermined depth, wherein the input data of the machine learning algorithm (212) comprises the one or more features and, optionally, also comprises the one or more ablation dependent variables; and
an output configured to provide the classification result.

The output may be configured to provide the classification result in real time and/or to a user and/or other processing system for further use.

The systems and methods of the current disclosure make available an indicator of lesion depth relative to a pre-determined depth to be used for various purposes. For example, the output may be suitable for connection to a user interface capable of receiving the data and providing a representation of the indicator to a user. Alternatively, or additionally, the output may be configured such that the data is available for the processor and/or another processor in further methods to be performed which methods benefit from or rely on the indicator.

Embodiments proposed by the present disclosure generate an indicator that indicates (e.g. in binary, numeric or categorical form) whether or not a depth of the lesion resulting from an ablation (e.g. an ongoing ablation or a completed ablation) reaches or exceeds a predetermined depth. The predetermined depth may set or chosen relative to the depth of a tissue undergoing ablation, e.g. it may be set equal to the depth of the tissue undergoing ablation or equal to the depth of the tissue undergoing ablation minus a predetermined value (e.g. 0.6 mm) or percentage (e.g. 10%). Information on the depth of the tissue may be provided by a user input and/or using an automated depth identification technique (e.g. that processes image data of the tissue and/or processes electrical signals from electrodes in the vicinity of the tissue). In other examples, the predetermined depth may be responsive to a user input, e.g. a user-specified depth.

The predetermined depth may be used to base labelling of the training data on in such a way that the algorithm is capable of classifying a lesion, based on input data, to be above or below the predetermined lesion depth.

Where the predetermined depth is equal to the depth of the tissue undergoing ablation, the classification result effectively provides an indicator of whether or not the lesion is transmural or not.

In the context of the present application, features that “correlate” with a depth are features that are responsive and/or sensitive to a change in depth of the lesion resulting from ablation. In particular, the features may be ones that represent some physical property or parameter of the tissue that is sensitive to changes in the depth of a lesion in the tissue.

In embodiments of the processing system the ablation dependent variables comprise or consist of: one or more of the voltages measured at the at least two electrodes, one or more of the currents measured at the at least two electrodes, or one or more local impedance values between pairs of the at least two electrodes. Preferably, the ablation dependent variables comprise or consist of: an electrical signal (such as for example a voltage or current) from the ablation electrode (104) and one or more local impedance values (208) between pairs of the at least two electrodes; There is also provided a system for determining whether an ablation of tissue (108) of a subject performed using an ablation catheter (102) creates a lesion having a depth that reaches or exceeds a predetermined depth, comprising:
the processing system according to any one of the processing systems as defined herein; and
   one or more of:
the ablation catheter (102) and
a user interface having an input for receiving the classification result from the processing system and for providing the classification result to the user of the system, wherein the user interface preferably comprises a display unit for providing a visual indication of the classification result and/or a speaker for providing an audible indication of the classification result and/or a device for providing a tactile indication of the classification result.

Ablation catheters have at least one ablation electrode (to perform tissue ablation) and reference electrodes. While typically at least the ablation electrode is used to generate electric field for ablation, in principle each of the other electrodes can also be used for that purpose. These electric fields can also be measured by any one of the electrodes. The electrical signals, such as e.g. voltage readings or current readings, from at least the ablation electrode contain information from the electric field which has reflected from the tissue being ablated. For example, the amount of reflected electric field changes with the extent of lesion depth, whether or not taken as absolute value or relative value with respect to a thickness of tissue the lesion is part of, or the extent of transmurality of the ablation lesion creation.

There is also a local impedance between each pair of the electrodes which is dependent on the local environment of the electrodes. It appears that during ablation, the local impedance values and the electrical signals on which they are based change with the development of the lesion and/or the temperature change of the tissue into changing depths.

Thus, the electrical signals received from the electrodes contains useful information on the extent of lesion depth, whether or not relative to a tissue thickness, or transmurality of the ablation. This useful information is then represented by so called ablation dependent variables comprised by the electrical signals, where an ablation dependent variable is an electrical characteristic which changes in dependence on the ablation. While some of these are directly measured, i.e. the raw signals such as the voltages or currents measured at the electrodes, some may be determined rather than directly measured, such as for example the impedances between electrode pairs. Thus, the ablation-dependent variables comprise basic electrical characteristics such as currents, voltages or impedances that are dependent on a state of ablation during an ablation procedure.

It has been observed that, training a machine learning algorithm with a complete set of the ablation-dependent variables, although possible, creates uncertainty in the algorithm output due to unknown freedom degrees. Therefore, features are derived from these ablation-dependent variables which preferably have an improved correlation with the extent of lesion propagation or advancement along a thickness direction of a tissue such as for example an extent of transmurality of an ablation. The derived features are then used for the training of a machine learning algorithm. These features are chosen such that they relate to the extent of lesion propagation of the ablation and may depend on the equipment used, the personal preference of the user etc. The features comprise combinations and/or adaptations of those ablation dependent variables in order to enhance the machine learning function. The features may also be referred to as ablation-dependent features. Examples of features include: the reactance(s) (imaginary component of impedance), high frequency component of the reactance(s), ratio of electrical signal at the beginning of ablation to current electrical signal, difference of the phase of the signal at the beginning of ablation to current electrical signal etc.

The machine learning algorithm is configured to output a classification result that comprises an indication of whether an ablation has reached or exceeded some predetermined depth, for example is transmural or non-transmural at any point in time during an ablation procedure. It does so based on the input data it is provided with. The input data include the features derived from the ablation-dependent variables. The features may be fed into the machine learning algorithm in real time, such that the classification is periodically and/or constantly updated and may additionally be periodically and/or even constantly output to a user of the system. In this respect the term “in real time” means that updating and/or outputting occurs at least once and preferably multiple times during an ablation procedure. In this way the evolvement of lesion formation during the ablation can be followed and the ablation procedure may be stopped at an appropriate time when an appropriate depth has been reached so that further lesion formation beyond the desired lesion formation may be reduced or even prevented from occurring.

The system may further comprise a memory for storing historic values for one or more of the ablation-dependent variables and/or features, wherein the input data of the machine learning algorithm may further comprise the historic values. Thus, the historic values of the electrical signals and local impedance values may also enable historic values of the features to be determined and used by the machine learning algorithm. By also using the historic values of the electrical signal and the local impedance values from the ablation procedure, a more accurate indication of whether the ablation has reached or exceeded the predetermined depth can be obtained. For example, all the data from a current defined time period (e.g. last second) of the ablation procedure can be input into the machine learning algorithm. For example, a running time window with defined time period comprising such electrical signals and impedances may be used.

The machine learning algorithm can be trained to detect patterns in the evolution of the electrical signals and the local impedance values throughout the ablation procedure which indicate that the ablation has reached or exceeded the predetermined depth.

The ablation-dependent variables may comprise a real component and an imaginary component, and wherein the features are one or more of
the ratio of the real component of an ablation-dependent variable, at the start of ablation, to the real component of the ablation-dependent variable, at the latest available time during ablation;
the ratio of the imaginary component of an ablation-dependent variable, at the start of ablation to the imaginary component of the ablation-dependent variable, at the latest available time during ablation;
the difference between the real component of an ablation-dependent variable, at the start of ablation, and the real component of the ablation-dependent variable, at the latest available time during ablation; and
the difference between the imaginary component of an ablation-dependent variable, at the start of ablation and the imaginary component of the ablation-dependent variable, at the latest available time during ablation.

The features may comprise one or more of
the average value of the ratios of the real components of an ablation-dependent variable over time;
the average value of the ratios of the imaginary components of an ablation-dependent variable over time;
the average value of the differences between the real components of an ablation-dependent variable over time; and
the average value of the differences between the imaginary components of an ablation-dependent variable over time.

The features may comprise one or more of
the ratio of the real component of an ablation-dependent variable, at the start of ablation, to the real component of the ablation-dependent variable, at the latest available time during ablation plus the real component of an ablation-dependent variable, at the start of ablation;
the ratio of the imaginary component of an ablation-dependent variable, at the start of ablation, to the imaginary component of the ablation-dependent variable, at the latest available time during ablation plus the imaginary component of an ablation-dependent variable, at the start of ablation;
the ratio of the real component of an ablation-dependent variable, at the start of ablation, to the real component of the ablation-dependent variable, at the latest available time during ablation minus the real component of an ablation-dependent variable, at the start of ablation; and
the ratio of the imaginary component of an ablation-dependent variable, at the start of ablation, to the imaginary component of the ablation-dependent variable, at the latest available time during ablation minus the imaginary component of an ablation-dependent variable, at the start of ablation.

A third-degree polynomial may be adapted to one or more of the ablation-dependent variables over time, and the features may be one or more of the polynomial coefficients for the one or more ablation-dependent variables.

The features may be one or more of:
the average value of the ratios of the absolute value of an ablation-dependent variable, at the start of ablation, to the absolute value of the ablation-dependent variable, at the latest available time during ablation, over time; and
the average value of the differences between the absolute value of an ablation-dependent variable, at the start of ablation, and the absolute value of the ablation-dependent variable, at the latest available time during ablation, over time.

An exponential function may be fitted to one or more admittance curves, wherein the admittance curves are based on the one or more impedance values over time, and wherein one or more of the exponential coefficients of the one or more exponential functions may be a feature.

The processor may be further configured to calculate the reflected energy from the tissue, wherein the reflected energy from the tissue is calculated based on the electrical signal and the impedance between the two closest electrodes to the tissue, and wherein the reflected energy is a feature.

Ablated tissue may reflect more energy from the ablation electrode than non-ablated tissue. This is due to the properties of the ablated tissue changing from the non-ablated tissue. The machine learning algorithm can be trained to detect the changes in the reflected energy and therefore more accurately indicate whether the ablation has created a lesion having a depth that reaches or exceeds the predetermined depth.

The input data of the machine learning algorithm may further comprise the duration of ablation. It may be desirable to be aware of the time of ablation, as the longer the ablation takes place, the more likely it is for the tissue lesion to reach or exceed the predetermined depth. The machine learning algorithm can be trained to be aware of how much time ablation procedures usually take. Therefore, by using the time of ablation as a further input, a more accurate indication of whether the lesion resulting from the ablation has reached or exceeded a predetermined depth can be obtained.

The input data of the machine learning algorithm may further comprise the thickness of the tissue. Previous measurements of the thickness of the tissue can be input into the machine learning algorithm which may help determine an accurate classification of whether the lesion resulting from the ablation has reached or exceeded a predetermined depth.

The machine learning algorithm may output a likelihood indicator, wherein the likelihood indicator indicates how likely it is for the ablation to be transmural. This likelihood indicator may act as the classification result provided by the machine learning algorithm.

For example, the machine learning algorithm could output a number from 0 to 1, 0 to 100, 1 to 100 (or any other number range, text range, color range etc.). The higher the number which is output, the higher the likelihood of the lesion depth having reached or exceeded the predetermined depth, e.g. of being transmural. This could allow the user to see when the lesion depth is close to being or exceeding the predetermined depth and allow for quicker reaction time when the lesion resulting from the ablation does reach the predetermined depth as the user is already aware that they will have to stop the ablation in a short period of time. This also provides useful clinical information after the completion of ablation, to assess the success of ablation and the likelihood of a desired lesion being generated.

The system may comprise a user interface having an input for receiving the classification result from the processing system and for providing the classification result to the user of the system, wherein the user interface preferably comprises a display unit for providing a visual indication of the classification result and/or a speaker for providing an audible indication of the classification result and/or a device for providing a tactile indication of the classification result.

Preferably the user interface comprises a display unit, wherein the display unit is configured to display a map of the tissue being ablated, and the classification result. Preferably the classification result from the machine learning algorithm is displayed above the tissue being ablated and shown on the map.

The memory could store the classification for one or more ablation zones, where ablation has been performed previously. Each ablation zone can then be displayed on the map of the tissue with the corresponding classifications.

According to examples in accordance with an aspect of the disclosure there is provided a method for determining whether an ablation of tissue performed by an ablation catheter (102) creates a lesion having a depth that reaches or exceeds a predetermined depth, wherein the ablation catheter (102) comprises at least two electrodes, wherein one of the electrodes is an ablation electrode (104) and the other electrode or electrodes are reference electrodes (106), wherein during the ablation, electrical signals are measured at the at least two electrodes which electrical signals comprise one or more ablation dependent variables, the method comprising:
receiving data comprising the one or more ablation-dependent variables;
deriving one or more features (210) from the received one or more ablation dependent variables, the one or more features (210) correlating with a depth of the lesion resulting from the ablation;
generating an ablation classification result using a machine learning algorithm (212) trained to provide the classification result based on an input, the classification result comprising an indication on whether or not the ablation a depth of the lesion resulting from the ablation reaches or exceeds a predetermined depth, wherein the input data of the machine learning algorithm (212) comprises the one or more features and, optionally, also comprises the one or more ablation dependent variables; and

optionally, providing, the classification result.

Providing the classification result may comprise providing the classification result in real time and/or to a user.

In the method the ablation dependent variables may comprise or consist of: one or more of the voltages measured at the at least two electrodes, one or more of the currents measured at the at least two electrodes, or one or more local impedance values between pairs of the at least two electrodes. Preferably, the ablation dependent variables comprise or consist of: an electrical signal (such as for example a voltage or current) from the ablation electrode (104) and one or more local impedance values (208) between pairs of the at least two electrodes;

In embodiments of the method the ablation-dependent variables comprise a real component and an imaginary component, and the one or more features (210) are one or more of:
the ratio of the real component of an ablation-dependent variable, at the start of ablation, to the real component of the ablation-dependent variable, at the latest available time during ablation;
the ratio of the imaginary component of an ablation-dependent variable, at the start of ablation to the imaginary component of the ablation-dependent variable, at the latest available time during ablation;
the difference between the real component of an ablation-dependent variable, at the start of ablation, and the real component of the ablation-dependent variable, at the latest available time during ablation; and
the difference between the imaginary component of an ablation-dependent variable, at the start of ablation and the imaginary component of the ablation-dependent variable, at the latest available time during ablation.

In embodiments of the method as defined herein before the one or more features (210) are one or more of:
the average value of the ratios of the real components of an ablation-dependent variable over time;
the average value of the ratios of the imaginary components of an ablation-dependent variable over time;
the average value of the differences between the real components of an ablation-dependent variable over time; and
the average value of the differences between the imaginary components of an ablation-dependent variable over time.

In embodiments of the method, there are used historic values for the one or more of the ablation-dependent variables, wherein the input data further comprises the historic values.

In embodiments of the method a decay function is adapted to one or more of the ablation-dependent variables over time, and the one or more features (210) comprise or consist of one or more of the coefficients of the decay function for the one or more ablation-dependent variables.

In embodiments of the method the one or more features (210) comprise or consist of one or more of:
the average value of the ratios of the absolute value of an ablation-dependent variable, at the start of ablation, to the absolute value of the ablation-dependent variable, at the latest available time during ablation, over time; and
the average value of the differences between the absolute value of an ablation-dependent variable, at the start of ablation, and the absolute value of the ablation-dependent variable, at the latest available time during ablation, over time.

In some embodiments, the method further comprises determining exponential coefficients by fitting an exponential function to one or more admittance curves, wherein the admittance curves are based on the one or more local impedance values (208) over time, and wherein the one or more features (210) comprise or consist of the exponential coefficients.

In embodiments of the method, the method further comprises determining the reflected energy and/or power from the tissue, wherein the reflected energy and/or power are calculated based on the electrical signal (204) and the local impedance between the two electrodes of the at least two electrodes that are closest to the tissue (preferably these include at least the ablation electrode), and wherein the one or more features (210) comprise or consist of the reflected energy and/or power.

In embodiments of the method, the input data of the machine learning algorithm (212) further comprises one or more of:
the duration of ablation;
the ablation generator power; and
the ablation generator electrical current.

In embodiments of the method the input data of the machine learning algorithm (212) further comprises the thickness of the tissue.

In embodiments of the method the one or more features (210) comprise or consist of the average value of the absolute difference of the phase of an ablation-dependent variable, at the start of ablation, and the phase of the ablation-dependent variable, at the latest available time during ablation, over time.

In embodiments of the method the one or more features (210) comprise or consist of the value of the area over a local impedance curve during ablation to a baseline impedance value line, wherein the local impedance curve is based on local impedance values over time.

In embodiments of the method the classification result further comprises a likelihood indicator indicating how likely it is for the depth of the lesion resulting from the ablation to reach or exceed the predetermined depth. In this case the method includes determining a likelihood indicator, e.g. as a classification result.

In embodiments of the method the classification result further comprises one or both of
a prediction of the dimensions of the ablation lesion caused by the ablation catheter (102); and
a prediction of steam pop and/or char occurrence during ablation.

In embodiments of the method, the method further comprises providing, in real time, the classification result to a user. For this a user interface may be used and preferably the user interface comprises a display unit for providing a visual indication of the classification result and/or a speaker for providing an audible indication of the classification result and/or a device for providing a tactile indication of the classification result.

For example, in the method a display unit may be used, wherein the display unit is configured to display a map of the tissue (108) being ablated, and the classification result, wherein preferably the classification result is displayed above or near the tissue.

The embodiments of the method described and claimed herein may be performed using a processing system as disclosed and defined herein. In particular, in the method the data may be received by the input of the system and the processor may be used to perform or orchestrate the method. The output possibly in combination with the user interface may be used to provide the classification result to the user.

The processor may be implemented in many ways as will be disclosed hereinafter.

According to examples in accordance with an aspect of the disclosure there is provided a computer program (product) comprising instructions or code for implementing any one of the methods disclosed when said program is run on a processor. The instructions or code is readable by a computer or processor.

The disclosure also provides a computer readable medium comprising the computer program (product) defined herein. The medium may be a non-transitory computer readable medium.

The processing system may include a memory for storing the instructions or code. The stored instruction and code may thus be used by the processor to perform any one of the methods.

These and other aspects of the disclosure will be apparent from and elucidated with reference to the embodiment(s) described hereinafter.

### BRIEF DESCRIPTION OF THE DRAWINGS

For a better understanding of embodiments of the invention, and to show more clearly how it may be carried into effect, reference will now be made, by way of example only, to the accompanying schematic drawings, in which:

FIG. 1 shows an ablation catheter performing an ablation;

FIG. 2 shows a flow diagram for determining whether an ablation lesion is transmural; and

FIG. 3 shows the evolution over time of an ablation lesion;

FIG. 4 is a flowchart illustrating a method;

FIG. 5 is a schematic diagram of a processor system 500 according to embodiments of the present disclosure; and

FIG. 6 shows a schematic of a catheter navigation and treatment system employing the processing system of disclosed embodiments.

### DETAILED DESCRIPTION OF THE EMBODIMENTS

The invention will be described with reference to the Figs.

It should be understood that the detailed description and specific examples, while indicating exemplary embodiments of the apparatus, systems and methods, are intended for purposes of illustration only and are not intended to limit the scope of the disclosure. These and other features, aspects, and advantages of the apparatus, systems and methods of the present disclosure will become better understood from the following description, appended claims, and accompanying drawings. It should be understood that the Figs. are merely schematic and are not drawn to scale. It should also be understood that the same reference numerals are used throughout the Figs. to indicate the same or similar parts.

The disclosure provides a controller, system and method for determining whether an ablation performed by an ablation catheter creates a lesion having a depth reaching or exceeding a predetermined depth, e.g. whether the lesion is transmural or not. The system comprises an ablation catheter with at least two electrodes for performing an ablation on tissue. One of the electrodes is an ablation electrode and the other electrode or electrodes are reference electrodes. A processor is used to determine, e.g. in real time, whether the ablation lesion depth has reached or exceeded the predetermined depth based on ablation-dependent variables. The ablation-dependent variables may comprise an electrical signal received by the ablation electrode and one or more impedance values between the electrodes. A machine learning algorithm can thus be used to classify the tissue lesion state in terms of whether or not the lesion is transmural or not. The inputs, i.e. the input data, of the machine learning algorithm are features derived from the ablation-dependent variables, wherein the features correlate with the state of a lesion and in particular with the depth of a lesion resulting from the ablation. The machine learning algorithm is adapted to output, e.g in real time, a classification comprising an indication of whether or not a depth of the lesion resulting from the ablation reaches or exceeds a predetermined depth, for example of whether or not a lesion is transmural or not. The classification and/or indication can be an index or identifier representative of whether or not the lesion is transmural.

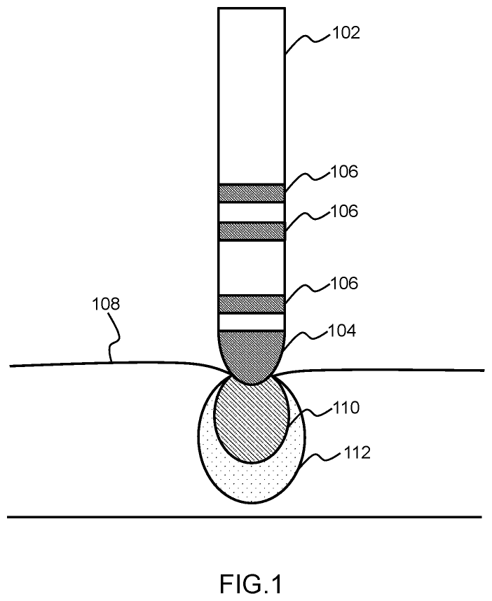

FIG. 1 shows an ablation catheter 102 performing an ablation. Ablation catheters 102 are well known devices used to perform ablation on a target tissue 108. Ablation catheters typically have an ablation electrode 104 at the tip and reference electrodes 106 around the shaft of the ablation catheter 102. In this example, a radio frequency ablation catheter 102 is used to burn a target tissue 108. A cross section of the tissue 108 can be seen. The evolution of the lesion caused by the ablation catheter can be seen in the cross section. The lesion spreads though the tissue, and the properties of the tissue may depend, for example, on the temperature rise of the tissue caused by the RF energy.

The larger section 112 shows an area of the tissue 108 which has been heated up, but has not become ablated yet. There is also a smaller section of necrotized tissue 110 which has already become ablated. The necrotized tissue 110 now has different properties to before, one of which is the necrotized tissue 110 has better electrical conductivity, thus the impedance typically drops during ablation due to extravasation of fluids from the cells (thus improving conductivity). Additionally, the necrotized tissue 110 will not return to its normal ability to conduct activation potentials between cells in the tissue after the ablation catheter 102 has been removed and it can cool down.

For cardiac arrhythmia, it may be desirable to create a (transmural) wall of necrotized tissue 110 between two areas of tissue 108, which will later develop into scar tissue, in order to prevent or reduce unwanted activation pathways from causing arrhythmias.

Each one of the electrodes injects a voltage with a high impedance connected to the channel at a specific frequency. This effectively creates a controlled current source injected into the body. The current waveform at each electrode may be at a different frequency for each one of the electrodes. This current source creates an electric field which interacts with the cardiac tissue. The electric field from each one of the electrodes may be measured as an electric potential (i.e. a voltage measurement) by any of the electrodes, and thus an electrical signal can be received at each one of the electrodes. Due to orthogonality of frequencies there is a separation of readings for each frequency created by each electrode. The building blocks for analysis of electrical behavior are the local readings of two or more electrodes at two or more of these frequencies. For a catheter having four electrodes, each of which provides a current of a different frequency and for each of which a voltage reading at each frequency is measured, there are 16 complex (magnitude and phase) voltage readings annotated as Vij, where i corresponds to the receiving electrode and j corresponds to frequency. The term “electrical signal” refers to the signals received by an electrode (e.g. V12 would be reading by electrode number 1 of frequency number 2 (created by electrode 2)).

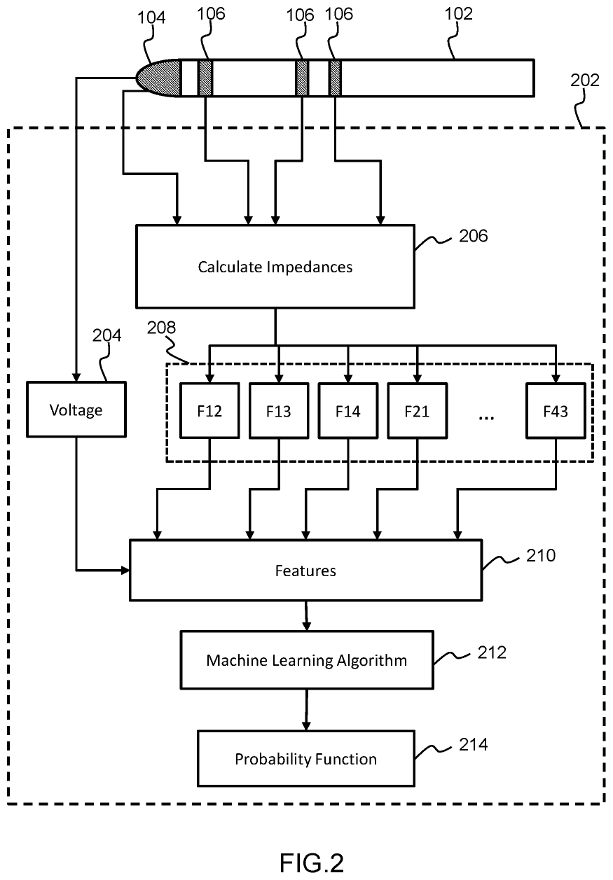

FIG. 2 shows a flow diagram for determining whether an ablation lesion has reached or exceeded a predetermined depth. Depending upon the predetermined depth, this may comprise determining whether an ablation lesion is transmural. Ablation catheters 102 typically comprise a plurality of electrodes. One of the electrodes (at the tip) is the ablation electrode 104, and the others can be referred to as reference electrodes 106. The ablation electrode 104 provides radiofrequency energy (in the case of RF ablation) in order to create the lesion and also can be used to transmit electric fields and receive electrical signals. The reference electrodes 106 are also used to transmit electric fields and receive electrical signals. Region 202 represents a processor, which performs the steps 204, 206, 208, 210, 212, 214 described below.

The electrical signals provide important information on the whether the depth of the lesion created has reached or exceeded a pre-determined depth, e.g. whether or not the lesion is transmural. However, this information is not easily accessed from the electrical signals received by the electrodes. In order to extract the important information, the impedance 208 between each pair of the electrodes is calculated based on the electrical signals from the electrodes. In FIG. 2, there are four electrodes shown, one of which is an ablation electrode 104 (at the tip). The impedances 208 are calculated with respect to each one of the electrodes, thus there are 12 possible impedances from the four electrodes.

The variables used may be the voltage readings 204 from the ablation electrode (transmitted electric field based on injected current and received electrical signal) and the impedances 208 calculated between each of the electrodes (F12, F13, F14, F21 . . . F43). However, in order for a machine learning algorithm 212 to learn when ablated tissue has reached or exceeded the predetermined depth (e.g. is transmural), it requires careful choice of inputs.

Thus, the inventors have derived a set of features 210 from the ablation-dependent variables which, when used as inputs (e.g. form input data), allow a machine learning algorithm 212 to classify or determine whether the depth of the lesion has reached or exceeded the predetermined depth, e.g. classify the ablated tissue as transmural/non-transmural. The meaning of “derived” in the context of the disclosure is that data is obtained from different initial data. The following example features (for a variable V) were chosen by the inventors in order for a machine learning algorithm 212 to learn whether a lesion depth has reached or exceeded the predetermined depth:

### FEATURES

(i) The ratio of the real initial value of V to its real value at the end of the transmission and reception interval (during ablation).
(ii) The ratio of the imaginary initial value of V to its imaginary value at the end of the transmission and reception interval (during ablation).
(iii) The difference of the real initial value of V to its real value at the end of the transmission and reception interval (during ablation).
(iv) The difference of the imaginary initial value of V to its imaginary value at the end of the transmission and reception interval (during ablation).
(v) The average value of the ratio of the real V initial value to the time-cumulative interval.
(vi) The average value of the ratio of the imaginary V initial value to the time-cumulative interval.
(vii) The average value of the ratio of the imaginary V initial value to the time-cumulative interval.
(viii) The average value of the difference between the imaginary V initial value and the time-cumulative interval.
(ix) Adapting a third-degree polynomial function to V. The polynomial coefficients, all four, may be used as features.
(x) The average value of the ratio of the absolute V initial value to the time-cumulative interval.
(xi) The average value of the difference between the absolute V initial value and the time-cumulative interval.
(xii) The average value of the ratio of the absolute value of an impedance (e.g. F12) to the absolute value of a different impedance (e.g. F21).
(xiii) The average value of the multiplication of the absolute value of an impedance (e.g. F12) and absolute value of a different impedance (e.g. F21).
(xiv) The standard deviation of the ratio of the absolute value of an impedance (e.g. F12) to the absolute value of a different impedance (e.g. F21).
(xv) The average value of the absolute difference between the phase of V, at the start of ablation, and the phase of the ablation-dependent variable, at the latest available time during ablation, over time.
(xvi) The average value of the absolute ratio of the phase of V, at the start of ablation, to the phase of the ablation-dependent variable, at the latest available time during ablation, over time.
(xvii) Adapting a mono-exponential model to the admittance curve I=1/F (of any one or more of the impedances F12, F13, F14, F21 . . . F43) during ablation. The mono-exponential model is composed of three coefficients: y(I)=A₀+A₁(1−e^{-τ·I}). The coefficients of the model may be used as features.
(xviii) The power used during ablation.
(xix) The duration of the ablation.
(xx) Ratios between different impedance values.

For the purpose of brevity, some features have been described as dependent on a variable V. The variable V may be any of the variables previously described, e.g. a voltage measurement at a particular electrode at a frequency of an electric field generated by any of the electrodes, and the features of all available variables may be used. Additionally, in the case where there is more (or less) electrodes in the ablation catheter 102, the impedance values between any two of the electrodes may be used as a further variables.

Any choice and/or combination of the previously mentioned or following features 210 may be used to train a machine learning algorithm 212 to determine whether an ablation lesion has a depth that reaches or exceeds the predetermined depth (or not), e.g. whether the ablation lesion is transmural or not. The choice and/or combination of features 210 may depend on the training data available, the judgement of the person skilled in the art, the machine learning algorithm used or ablation catheter 102 used.

Additionally, a model can be created with a large set of features and the Random Forest method can be used to determine which features 210 to use to train the machine learning algorithm 212. Alternatively, the size of the feature coefficient/weighting can be used to determine which features 210 to use.

In an example, the following features 210 may be used to train a machine learning algorithm 212 to determine whether the depth of the ablation lesion has reached or exceeded the predetermined depth:

Feature 1—The difference between the magnitudes of consecutive frames in the voltage magnitude signal (VMS) is calculated for the first catheter electrode (ablation electrode) during the ablation and a differences vector is created.

A condition-based threshold is thus applied to the differences vector and, based on the threshold, the difference vector is converted into a binary vector. The calculated binary vector is summed and the result is divided by the duration of the VMS. The divided vector is thus Feature 1.

Feature 2—The length (in frames) of the VMS is divided by the sampling rate (e.g. 100 Hz). This gives Feature 2, which represents the duration in seconds of the ablation.

Feature 3—The ratio of (i) the average of the VMS during one second (e.g. 100 frames) which follows the first second of the ablation to (ii) the average of the VMS during one second (e.g. 100 frames) that precedes the latest (available) second of the ablation. (E.g. final second of ablation).

Feature 4—The difference between (i) the average of the VMS during one second (100 frames) which follows the first second of the ablation to (ii) the average of the VMS during one second (100 frames) that precedes the latest (available) second of the ablation. (E.g. final second of ablation)

Features 5 & 6—The impedance values between electrodes are calculated using the VMS received in all catheter electrodes and the ratio of (i) the average value of the impedance values in the first 0.5 seconds (e.g. 50 frames) of the ablation to (ii) the average value of the impedance values in the latest (available) 5 seconds (e.g. 500 frames) of the ablation is further calculated. Feature 5 is the real component of the calculated ratio and feature 6 is the imaginary component of the calculated ratio.

Features 7 & 8—The impedance values between electrodes are calculated using the VMS received in all catheter electrodes and the difference between (i) the average value of the impedance values in the first 0.5 seconds (e.g. 50 frames) of the ablation and (ii) the average value of the impedance values in the latest (available) 5 seconds (e.g. 500 frames) of the ablation is further calculated. Feature 7 is the real component of the calculated difference and Feature 8 is the imaginary component of the calculated difference.

Feature 9—An exponential function ƒ(t)=a·e^{bt}+c is fitted to the VMS (during ablation for real time measurement or post ablation for checking whether the depth of a lesion has reached or exceeded a predetermined threshold depth). Feature 9 is coefficient b.

Feature 10—The reflected energy from the tissue during/post the ablation period is determined by calculating the electrical signal VMS (received in the ablation electrode) squared value (per frame) and calculating the impedance Z between the ablation electrode and a reference electrode. The reflected energy can thus be calculated from the equation:

$$Reflected Energy = \int \frac{{VM}^{2}}{Z} d t$$

A Support Vector Machine (SVM) may be used as the machine learning algorithm 212 as an appropriate classifier. Alternatively, any other known machine learning algorithms 212, such as neural networks, may be used to determine whether a lesion depth has reached or exceeded the predetermined depth based on the features 210. The machine learning algorithm 212 can thus be trained to output a probability function 214 which can itself be used as the classification result or can be further processed to be used to determine a category the ablated tissue (e.g. reached/exceeded predetermined thickness/near reaching/exceeding the predetermined thickness/has not reached/exceeded the predetermined thickness).

An SVM can be trained to generate a classification result (based on the features 210) that indicates whether or not a lesion has a depth that reaches or exceeds the predetermined depth, e.g. whether the lesion is transmural or non-transmural. In some embodiments the predetermined depth is set during labelling of the training data as will be explained in more detail below. Additionally, the SVM can be trained to provide additional information on the lesion such as the time before the lesion reaches or exceeds the predetermined depth, the probability of unwanted events (e.g. steam pop) etc.

An important property of some of the features 210 above is that they include time-continuous information of the ablation lesion. Different tissue walls will have numerous different properties and may take longer/shorter to become transmural during ablation depending on the numerous different properties (e.g. thickness, age, muscle composition, blood flow, temperature, ablation catheter used etc.).

However, the time evolution of some of the features mentioned allows a more accurate estimation of when the ablation lesion has reached or exceeded the predetermined depth. The time-dependent features (e.g. adapted third degree polynomial, mono-exponential model etc.) in combination with the time independent features (power, average values, ratios, differences etc.) allows a machine learning algorithm 210 to learn the evolution of ablation lesions, and thus, when they reach or exceed the predetermined depth.

Additional inputs to the machine learning algorithm 212 may be: the thickness of the tissue wall (if known); ablation generator power, ablation generator temperature and/or tissue pressure from the ablation catheter.

The predetermined depth may be a chosen responsive to a user input and/or a thickness of the tissue.

In one example, the predetermined depth may be equal to the thickness of the tissue undergoing ablation, so that the classification result effectively comprises an indicator of whether or not the ablation is completely transmural.

In another example, the predetermined depth may be equal to the thickness of the tissue minus a predetermined value/percentage, e.g. minus 0.6 mm or minus 10%. The predetermined value may, for instance, be selectable be a user (e.g. via a user interface) or static, i.e. unchangeable by a user.

In any above example, the thickness of the tissue may be indicated via a user input (e.g. indicating the thickness of the tissue) or may be automatically determined, e.g. through appropriate processing of tissue-thickness dependent features derived from electrical signals of the electrodes on the catheter and/or processing/segmentation of medical image data of the tissue. Other thickness reference value databases can also be used.

In other examples, the predetermined thickness may be a user-selected or user-input thickness, e.g. input by a user via a user interface.

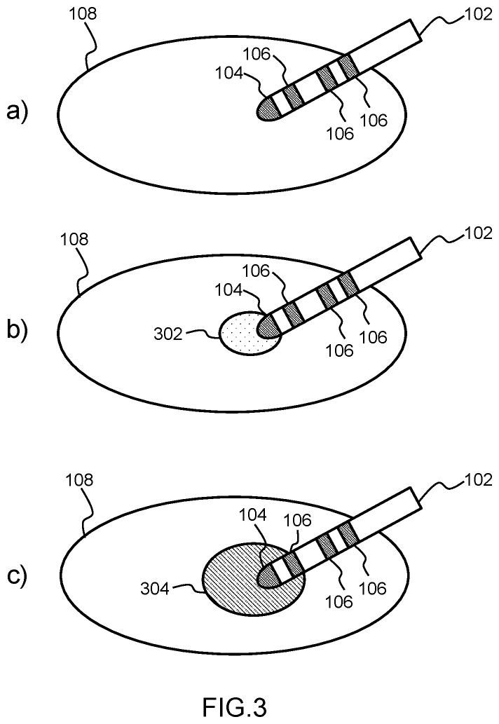

FIG. 3 shows the evolution over time of an ablation lesion.

An SVM or other machine-learning algorithm may be trained to output, as its classification result, a numeric indicator of a likelihood that a lesion resulting from the (ongoing or monitored) ablation has a depth that reaches or exceeds the predetermined depth. This numeric indicator may, for instance, be a probability on a scale or 0 to 1 or 0 to 100 (although other numeric scales would be readily apparent to the skilled person).

An SVM can be trained to output a classification of predetermined depth attainment (such as for example transmurality attainment) when RF delivery is completed (point-by-point method) or when the ablation catheter 102 is moved from the point (“dragging method”). Predetermined depth attainment can be represented by, for example, color coding of the ablation point (different colors represent lesions that have reached or exceeded the predetermined depth and lesions that have not reached or exceeded the predetermined depth), graphical image of lesions that have reached or exceeded the predetermined depth, text indication etc. However any other kind of indicator can be used to represent the classification output. These may be visible indicators on a user interface such as display and/or audible and/or tactile indicators.

The SVM may be trained to output, as the classification result, a (e.g. real-time) indication of lesion state classified to: “Not Reached/Exceeded Predetermined Depth”; “Near Reaching/Exceeding Predetermined Depth”; or “Reached/Exceeded Predetermined Depth”. In embodiments, when the predetermined depth distinguishes between transmural or non-transmural state of the lesion the classifier may indicate: Non transmural/Near Transmural/Transmural or any other wording and/or graphics/and or sounds to representative thereof. The lesion state can be represented as color coding of the point (e.g. Green-Yellow-Red), text indication, graph running on the screen changing from zero value to one, graph of real-time voltage/impedance value where the line can change color according to the lesion state.

Additionally, a graphical image representation of the ablation lesion dimensions may be shown, showing the graphical representation of the lesion expansion (balloon inflation like) to predict the lesion sizes during real time creation.

In FIG. 3 a) the ablation catheter 102 is shown being placed on the target tissue 108. As the ablation procedure begins, the ablation electrode 102 heats up the target tissue 108.

FIG. 3 b) shows an indication of the lesion resulting in the target tissue being near to reaching/exceeding the predetermined depth 302. Alternatively, the indication 302 could be a color, number, text or graphical image which indicates to the clinician that ablation is taking place, and that the lesion is near to reaching/exceeding the predetermined depth.

In FIG. 3 c) the target tissue 108 is shown in a state 304, in which the lesion induced in the tissue has reached/exceeded the predetermined depth, near the ablation catheter 102. The SVM indicates on a map of the target tissue 108, based on the input features 210, that the lesion created by the ablation catheter 102 is now in the state 304. This could indicate to the clinician to remove the ablation catheter 102 and/or turn it off, in order to prevent any unwanted damage to the tissue 108.

In some embodiments of FIG. 3 the indications of state 302 and 304 may refer to states of near transmurality and transmurality, respectively.

A class and a “probability function” may be calculated to indicate states of the depth of the lesion. The real-time indication of lesion state is based on a probability function 214 generated by a Support Vector Machine algorithm. Determination of the probability function threshold value that is used to indicate the point in time where the ablation procedure results in a lesion that reaches or exceeds the predetermined depth may be based on empirical results, findings and the SVM predications.

A machine-learning algorithm is any self-training algorithm that processes input data in order to produce or predict output data. Here, the input data comprises at least some of the features previously mentioned and the output data comprises an indication of whether the depth of a lesion resulting from ablation has reached/exceeded a predetermined depth or not.

Suitable machine-learning algorithms for being employed in the present disclosure will be apparent to the skilled person. Examples of suitable machine-learning algorithms include decision tree algorithms and artificial neural networks. Other machine-learning algorithms such as logistic regression, support vector machines or Naïve Bayesian models are suitable alternatives.

The structure of an artificial neural network (or, simply, neural network) is inspired by the human brain. Neural networks are comprised of layers, each layer comprising a plurality of neurons. Each neuron comprises a mathematical operation. In particular, each neuron may comprise a different weighted combination of a single type of transformation (e.g. the same type of transformation, sigmoid etc. but with different weightings). In the process of processing input data, the mathematical operation of each neuron is performed on the input data to produce a numerical output, and the outputs of each layer in the neural network are fed into the next layer sequentially. The final layer provides the output.

Methods of training a machine-learning algorithm are well known. Typically, such methods comprise obtaining a training dataset, comprising training input data entries and corresponding training output data entries. An initialized machine-learning algorithm is applied to each input data entry to generate predicted output data entries. An error between the predicted output data entries and corresponding training output data entries is used to modify the machine-learning algorithm. This process can be repeated until the error converges, and the predicted output data entries are sufficiently similar (e.g. ±1%) to the training output data entries. This is commonly known as a supervised learning technique.

For example, where the machine-learning algorithm is formed from a neural network, (weightings of) the mathematical operation of each neuron may be modified until the error converges. Known methods of modifying a neural network include gradient descent, backpropagation algorithms and so on. Approaches for training other forms of machine-learning algorithms, such as a support vector machine, are well established in the prior art.

For instance, one method of training support vector machine is disclosed by Osuna, Edgar, Robert Freund, and Federico Girosi. “An improved training algorithm for support vector machines.” Neural networks for signal processing VII. Proceedings of the 1997 IEEE signal processing society workshop. IEEE, 1997. Another approach is suggested by Catanzaro, Bryan, Narayanan Sundaram, and Kurt Keutzer. “Fast support vector machine training and classification on graphics processors.” Proceedings of the 25th international conference on Machine learning. 2008.

In a training step there is provided training data. The training input data entries correspond to example features of ablation lesions. The training output data entries correspond to indications of whether the depth of a lesion resulting from ablation has reached/exceeded a predetermined depth.

In one example, a portion of the training dataset may be generated using an in-vitro or an in-vivo approach. Samples of one or more suitable features for the machine-learning algorithm can be obtained using a catheter that is variably positioned about tissue (e.g. of a human or of a non-human animal/mammal, such as a pig) that has undergone a degree of ablation, with a respective (true) measure of lesion depth resulting from ablation for each sample of one or more suitable features being determined through a physician analysis (e.g. by a physician analyzing a medical image of the subject), an automated analysis (e.g. segmentation of a medical image of the subject and the lesion in question) or direct measurement, e.g. via tissue dissection to determine a lesion depth with respect to the wall thickness of the wall the lesion is in.

In some embodiments, a labelling of the training data set then includes choosing or setting a pre-determined lesion depth as a threshold for deciding whether a lesion fulfills the criterion of having reached the predetermined depth. For example, it may be decided that a lesion is to be labelled transmural when the lesion depth exceeds a percentage of the thickness of the tissue. Such percentage may be equal to any one of the following values: 50%, 60%, 70%, 80%, 90%. The predetermined depth may even be set at 100% of thickness.

The choice of pre-determined depth during labelling determines what the machine learning algorithm is trained to output and thus determines the state of the lesion with respect to the pre-determined depth in terms of reached or not reached, or in other words less deep or deeper, or when the pre-determined depth relates to a property distinguishing between transmural or not transmural an output that is indicative of the lesion being transmural or not. In such case the algorithm does not provide an actual lesion depth.

In some embodiments, a labelling of the training data set includes adding a relative lesion depth (for example in the form of ratio of lesion depth to tissue thickness) to the input data.

In some examples, a validation dataset may be used to check the accuracy and/or reliability of the trained machine-learning algorithm. Thus, the validation dataset may contain further examples of input data entries (for the machine-learning algorithm) and corresponding output data entries, which have not been used to train the machine-learning algorithm. The validation dataset may be used to determine an accuracy of the machine-learning network, e.g. by processing the input data entries of the validation dataset using the trained machine-learning algorithm and determining an accuracy of the data output by the machine-learning algorithm. The validation dataset may form part of a larger dataset containing the training dataset, e.g. comprising data obtained from a same source.

Where a larger dataset comprises the training dataset and the validation dataset, the training dataset may contain a predetermined percentage portion of the larger dataset, e.g. 99% of the larger dataset.

In particular example, the training dataset may contain input data entries obtained using a first set of tissue examples and the validation dataset may contain input data entries obtained using a second set of tissue examples, where each set of tissue examples contains at least one unique tissue example (i.e. not found in the other set). This helps facilitate assessment of a more appropriate accuracy of the machine-learning method for different subjects.

An algorithm may accordingly be trained for deciding from test (measured) data whether a lesion has reached or exceeded the predetermined depth. The parameters of the trained algorithm may be stored in a memory e.g. within a lookup table or in any other form for use during assessment of a lesion. The algorithm may be trained for multiple mutually different predetermined depths and for each these predetermined depths the set of optimized algorithmic parameters obtained after training may be stored in the memory. In some embodiments. Thus, a set of parameters may be chosen based on predetermined depth criterion desired. Such criterion may then be user defined or automatically system defined. For example, if a user desires lesions of a specific predetermined depth of e.g. 40% he can set the predetermined depth at this value or a nearby value and the system chooses the corresponding parameter set optimized during the training for this depth. During or after ablation the system can then tell the user whether or not he has created lesions with that desired thickness.

Training can commence using known in the art methods such as for example using minimization of a cost function by changing algorithmic parameters such as weights and biases used in the machine learning algorithm. Such procedures will not be describe here as they are entirely standard and generally known in the art.

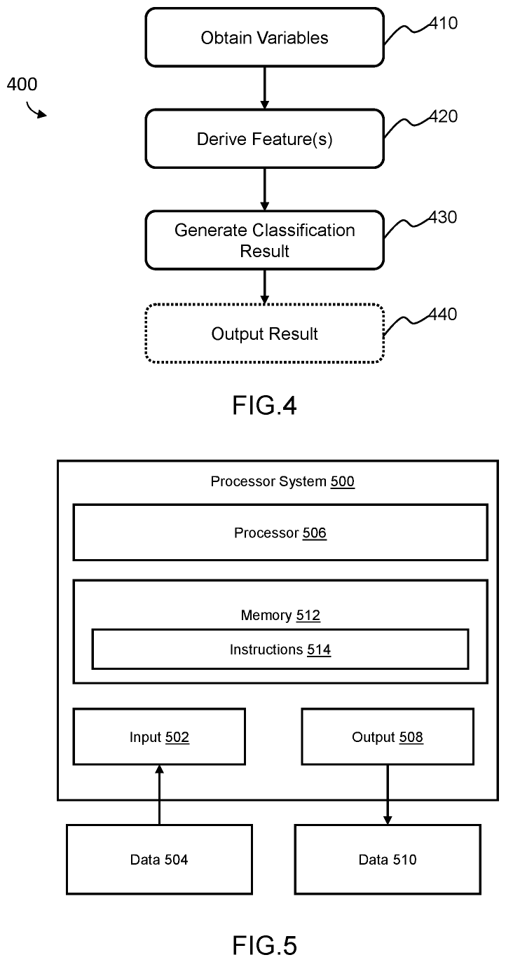

FIG. 4 is a flowchart illustrating a (computer-implemented) method 400 according to an embodiment. The method is for determining whether an ablation of tissue performed by an ablation catheter creates a lesion having a depth that reaches or exceeds a predetermined depth, wherein the ablation catheter comprises at least two electrodes, wherein one of the electrodes is an ablation electrode and the other electrode or electrodes are reference electrodes, wherein during the ablation, electrical signals are measured at the at least two electrodes which electrical signals comprise one or more ablation dependent variables.

The method comprises a step 410 of receiving data comprising the one or more ablation-dependent variables. The method also comprises a step 420 of deriving one or more features from the received one or more ablation dependent variables, the one or more features correlating with a depth of the lesion resulting from the ablation. The method also comprises a step 430 of generating an ablation classification result using a machine learning algorithm trained to provide the classification result by processing input data, the classification result comprising an indication on whether or not a depth of the lesion resulting from the ablation reaches or exceeds the predetermined depth, wherein the input data of the machine learning algorithm comprises the one or more features and, optionally, also comprises the one or more ablation dependent variables.

The method may comprise a step 440 of providing the classification result. This may comprise providing the classification result to a user (e.g. via the user interface) or to another processor or processing system.

Step 410 may be performed by an input of a processing system. Steps 420 and 430 may be performed by a processor of the processing system. If performed, step 440 may be carried out by an output of the processing system. The input, processor and output (if present) of the processing system may represent different electrical circuits of a processing system.

The skilled person would be readily capable of developing a processor for carrying out any herein described method. Thus, each step of a flow chart may represent a different action performed by a processor, and may be performed by a respective module of the processing processor.

As discussed above, the processing system makes use of processor to perform the data processing. The processor can be implemented in numerous ways, with software and/or hardware, to perform the various functions required. The processor typically employs one or more microprocessors that may be programmed using software (e.g., microcode) to perform the required functions. The processor may be implemented as a combination of dedicated hardware to perform some functions and one or more programmed microprocessors and associated circuitry to perform other functions.

FIG. 5 is a schematic diagram of a processor system 500 according to embodiments of the present disclosure. As shown, the processor system 500 includes an input 502 for receiving the data 504 that comprise the ablation dependent variables. The processing system further includes a processor 506 and an output 508 for outputting data 510 which comprise the classification result. Optionally the processing system has a memory 512 for storing code or instructions for performing steps of the methods disclosed herein. The elements 502, 506, 508 and optionally 512 may be in direct (wired) or indirect (wireless) communication with each other, for example via one or more communication buses (not shown). Examples of circuitry that may be employed in various embodiments of the processing system include, but are not limited to, conventional microprocessors, application specific integrated circuits (ASICs), and field-programmable gate arrays (FPGAs). The processor 506 may include a single central processing unit (CPU), a digital signal processor (DSP), an ASIC, a controller, an FPGA, another hardware device, a firmware device, or any combination thereof configured to perform the operations described herein and that may be encoded by the code and instructions stored in the memory 512. The processor 506 may also be implemented as a combination of computing devices, e.g., a combination of a DSP and a microprocessor, a plurality of microprocessors, one or more microprocessors in conjunction with a DSP core, or any other such configuration.

The memory 512 may include a cache memory (e.g., a cache memory of the processor 506), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory.

In embodiments, the memory 512 includes a non-transitory computer-readable medium. The memory 512 may store instructions 514. The instructions 514 may include instructions that, when executed by the processor 506, cause the processor 506 to perform the operations described herein such as the the steps defined by the methods as defined herein. Instructions 514 may also be referred to as code. The terms “instructions” and “code” should be interpreted broadly to include any type of computer-readable statement(s). For example, the terms instructions and code may refer to one or more programs, routines, sub-routines, functions, procedures, etc. Instructions and code may include a single computer-readable statement or many computer-readable statements. The memory may be encoded with one or more programs that, when executed on one or more processors and/or controllers, perform the required functions. Various memory types may be fixed within a processor or controller or may be transportable as with mobile storage media known in the art (Optical disc, magnetic disk, Flash or SD, such that the one or more programs stored thereon can be loaded into a processor.

The input 502 and output 508 can include any communications buses and/or electronic circuitry and/or logic circuitry to facilitate direct or indirect communication of data between the processor system and a system for generating the data 504 or for receiving the data 510.

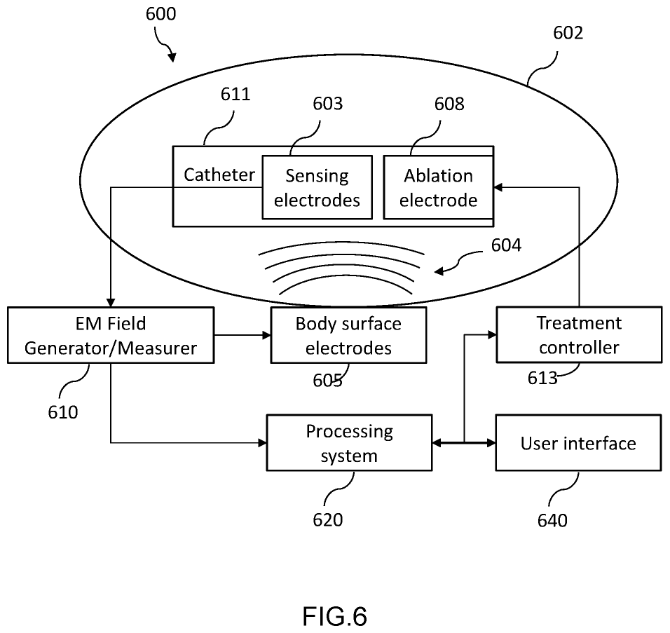

The processing system 500 may be used with or implemented in a system for ablating tissue of a subject. Embodiments of such system are described by way of example with reference to FIG. 6 which schematically represents a navigation and treatment system 500. Although in these embodiments there is also a navigation system, this is not necessary. The processing system may also be used only in combination with the treatment system. While treatment systems in the form of RF ablation systems including the catheters and signal generators are known in the art, for the purpose of description of implementation of the processing system and methods disclosed, the combined navigation and treatment system will be employed here.

The treatment and navigation system 600 of FIG. 6 is for treatment of a tissue 602 of a subject and includes the processing system 620 which may be the processing system described with reference of FIG. 65. The processing system further includes inputs and outputs for communicating with user interface 640. For example a user can input commands and data for a procedure to be performed. Furthermore, the user may be presented with outputs from the processing system such as the classification results and optionally navigation data including anatomical maps and the like. The system 600 further includes a electromagnetic (EM) field generator/measurer 610 and treatment controller 613 and these and the the processing system are configured to communicate over appropriate communication busses so that the processing system is capable of controlling the (EM) field generator/measurer 610 and treatment controller 613 to provide the appropriate signals to the catheter 611 and/or the body surface electrodes and is capable of receiving data (such as those measured by the catheter) from either of these devices. The system 600 is configured to obtain electro-anatomical data to generate three-dimensional electro-anatomical images of the anatomy (e.g., an interior of a heart).

In some embodiments, the system 600 is configured to induce or generate at least one time-varying electromagnetic (EM) field 604 (for example, three or more crossing electromagnetic fields, each of a different frequency) using an electromagnetic field generator/measurer 610 (which is optionally itself comprised of a plurality of field generation modules) using electrodes such as body surface electrodes 605 across a region of anatomy 602 that is targeted to be navigated by catheter 611. The catheter 611 may be in communication with the processing system 20, and can include a flexible elongate member sized, shaped, structurally arranged, and/or otherwise configured to be positioned within a body lumen of a patient, such as a blood vessel. In particular, the distal portion of the catheter can include electrodes, sensors, or other electronic components configured to be positioned within the body lumen to sense, image, and/or perform therapeutic procedures within the body lumen. A proximal portion of the catheter can be positioned outside the body of the patient, and can be coupled to an interface for electrical communication within the processing system 620 and/or EM field generator/measurer. The catheter can include one or more communication lines, such as electrical conductors and/or fiber optic cables, positioned within the flexible elongate member and configured to communicatively couple the electrodes, sensors, and/or other electronic components to the interface.

The catheter 611 includes a treatment electrode 608 (similar to the electrode 104 of FIG. 1) or ablation electrode controllable by the processing system 20 via the treatment controller to perform ablation on a region of tissue in the anatomy 602 and includes the sensing electrodes 603 (similar to the electrodes 106 of FIG. 1) which in this case are controlled by the processing system via the EM filed generator/measurer.

The electrodes of catheter 611 can thus be used by the system 600 to provide the processor system 620 with the required data for providing the classification by the method as describe herein before with reference to e.g. FIGS. 1, 2, 3 and 4.

In some embodiments, position data is acquired from the catheter probe 611, from each of a plurality (e.g., 2, 3, 4 or more) of sensing electrodes 603 on the probe which act as sensors to measure electromagnetic field data indicative of position. The electrodes 603 can be controlled by and/or in communication with the processing system 620 to obtain the electromagnetic field data, such as electro-anatomical data. In some embodiments of the disclosure, the sensing electrodes 603 are in a known spacing relative to one another; for example, fixed at certain distances from one another. Alternatively, if the sensing electrode 603 spacing is dynamic (e.g. because the probe 611 can bend), the spacing can be estimated to change in correlation with parameters of probe operation (e.g., active deformation) and/or measured contact (e.g., deformation correlated with measurements of contact force). The known spacing is used, in some embodiments, as part of the data used in the reconstruction of the body cavity (e.g., a lumen of a hollow organ such as a heart chamber) within which the intrabody probe moves. For example, the catheter probe 611 can be used within a blood vessel, heart chambers, and/or any other suitable body cavity or area of the anatomy to generate electro-anatomical images. In some embodiments, position data is received by computer circuitry, e.g., from the sensors in real time or from a computer memory that saves data received from the sensors.

Further details regarding generating electro-anatomical images using electrodes disposed on a catheter can be found in, for example, WO 2018/120974 to Dichterman, et al., the entirety of which is hereby incorporated by reference for all of its features and purposes.

Further details on devices and methods with which the systems and methods of the current disclosure may be configured to obtain tissue dependent variables for lesion assessment of an ablation procedure such as impedance data of tissue can be found in, for example, U.S. Publication No. 2018/0125575 to Schwartz, et al., the entirety of which is hereby incorporated by reference for all of its features and purposes.

The anatomical imaging data may be used to construct a map including the classification data and the combination may be provided to the user.

The skilled person would be readily capable of developing a processing system for carrying out any herein described method. Thus, each step of the flow chart may represent a different action performed by a processing system, and may be performed by a respective module of the processing system.

Embodiments may therefore make use of a processing system. The processing system can be implemented in numerous ways, with software and/or hardware, to perform the various functions required. A processor is one example of a processing system which employs one or more microprocessors that may be programmed using software (e.g., microcode) to perform the required functions. A processing system may however be implemented with or without employing a processor, and also may be implemented as a combination of dedicated hardware to perform some functions and a processor (e.g., one or more programmed microprocessors and associated circuitry) to perform other functions.

Examples of processing system components that may be employed in various embodiments of the present disclosure include, but are not limited to, conventional microprocessors, application specific integrated circuits (ASICs), and field-programmable gate arrays (FPGAs).

In various implementations, a processor or processing system may be associated with one or more storage media such as volatile and non-volatile computer memory such as RAM, PROM, EPROM, and EEPROM. The storage media may be encoded with one or more programs that, when executed on one or more processors and/or processing systems, perform the required functions. Various storage media may be fixed within a processor or processing system or may be transportable, such that the one or more programs stored thereon can be loaded into a processor or processing system.

It will be understood that disclosed methods are preferably computer-implemented methods. As such, there is also proposed the concept of a computer program comprising code means for implementing any described method when said program is run on a processing system, such as a computer. There is also proposed a (non-transitory) computer readable medium comprising the computer program. Thus, different portions, lines or blocks of code of a computer program according to an embodiment may be executed by a processing system or computer to perform any herein described method. In some alternative implementations, the functions noted in the block diagram(s) or flow chart(s) may occur out of the order noted in the figures. For example, two blocks shown in succession may, in fact, be executed substantially concurrently, or the blocks may sometimes be executed in the reverse order, depending upon the functionality involved.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. If a computer program is discussed above, it may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted the term “adapted to” is intended to be equivalent to the term “configured to”. Any reference signs in the claims should not be construed as limiting the scope.

Further examples according to the disclosure are provided in the form of the following CLAUSES:
Clause 1. A system for determining whether an ablation performed by an ablation catheter (102) is transmural, comprising:
   an ablation catheter (102) with at least two electrodes for performing an ablation on tissue (108), wherein one of the electrodes is an ablation electrode (104) and the other electrode or electrodes are reference electrodes (106);
   a processor (202) for determining, in real time, whether the ablation is transmural, wherein the processor (202) is configured to:
      determine ablation-dependent variables, wherein the ablation-dependent variables comprise:
         an electrical signal (204) from the ablation electrode (104); and
         one or more local impedance values (208) between the electrodes;
         and
      use a machine learning algorithm (212) to classify the tissue (108) which is being ablated, wherein the inputs of the machine learning algorithm (212) are features (210) derived from the ablation-dependent variables, wherein the features (210) correlate with the transmurality of an ablation, and wherein the machine learning algorithm (212) is adapted to output, in real time, a classification of whether the ablation is transmural or non-transmural.
Clause 2. The system according to Clause 1, further comprising a memory for storing historic values for one or more of the ablation-dependent variables, wherein further inputs of the machine learning algorithm (212) are the historic values.
Clause 3. The system according to Clause 1, wherein the ablation-dependent variables comprise a real component and an imaginary component, and wherein the features (210) are one or more of:
   the ratio of the real component of an ablation-dependent variable, at the start of ablation, to the real component of the ablation-dependent variable, at the latest available time during ablation;
   the ratio of the imaginary component of an ablation-dependent variable, at the start of ablation to the imaginary component of the ablation-dependent variable, at the latest available time during ablation;
   the difference between the real component of an ablation-dependent variable, at the start of ablation, and the real component of the ablation-dependent variable, at the latest available time during ablation; and
   the difference between the imaginary component of an ablation-dependent variable, at the start of ablation and the imaginary component of the ablation-dependent variable, at the latest available time during ablation.
Clause 5. The system according to Clause 4, wherein the features (210) are one or more of:
   the average value of the ratios of the real components of an ablation-dependent variable over time;
   the average value of the ratios of the imaginary components of an ablation-dependent variable over time;
   the average value of the differences between the real components of an ablation-dependent variable over time; and
   the average value of the differences between the imaginary components of an ablation-dependent variable over time.
Clause 6. The system according to Clause 1, wherein a decay function is adapted to one or more of the ablation-dependent variables over time, and wherein the features (210) are one or more of the coefficients of the decay function for the one or more ablation-dependent variables.
Clause 7. The system according to Clause 1, wherein the features (210) are one or more of:
   the average value of the ratios of the absolute value of an ablation-dependent variable, at the start of ablation, to the absolute value of the ablation-dependent variable, at the latest available time during ablation, over time; and
   the average value of the differences between the absolute value of an ablation-dependent variable, at the start of ablation, and the absolute value of the ablation-dependent variable, at the latest available time during ablation, over time.
Clause 8. The system according to Clause 1, wherein an exponential function is fitted to one or more admittance curves, wherein the admittance curves are based on the one or more local impedance values (208) over time, and wherein one or more of the exponential coefficients of the one or more exponential functions are features (210).
Clause 9. The system according to Clause 1, wherein the processor (202) is further configured to calculate the reflected energy from the tissue, wherein the reflected energy from the tissue is calculated based on the electrical signal (204) and the local impedance between the two closest electrodes to the tissue, and wherein the reflected energy is a feature (210).
Clause 10. The system according to Clause 1, wherein further inputs of the machine learning algorithm (212) are one or more of:
   the duration of ablation;
   the ablation generator power; and
   the ablation generator electrical current.
Clause 11. The system according to Clause 1, wherein a further input of the machine learning algorithm (212) is the thickness of the tissue.
Clause 12. The system according to Clause 1, wherein a feature (210) is the average value of the absolute difference of the phase of an ablation-dependent variable, at the start of ablation, and the phase of the ablation-dependent variable, at the latest available time during ablation, over time.
Clause 13. The system according to Clause 1, wherein a feature (210) is the value of the area over a local impedance curve during ablation to a baseline impedance value line, wherein the local impedance curve is based on local impedance values over time.
Clause 14. The system according to Clause 1, wherein the machine learning algorithm (212) further outputs a likelihood indicator, wherein the likelihood indicator indicates how likely it is for the ablation to be transmural.
Clause 15. The system according to Clause 1, wherein the machine learning algorithm (212) further outputs a prediction of the dimensions of the ablation lesion caused by the ablation catheter (102).
Clause 16. The system according to Clause 1, wherein the machine learning algorithm (212) further outputs a prediction of steam pop and/or char occurrence during ablation.
Clause 17. The system according to Clause 1, further comprising a display unit, wherein the display unit is configured to display a map of the tissue (108) being ablated, and wherein the classification from the machine learning algorithm (212) is displayed on the tissue (108) which has been classified.
Clause 18. A method for determining whether an ablation performed by an ablation catheter (102) is transmural, comprising:
   determining ablation-dependent variables, wherein the ablation-dependent variables comprise:
      an electrical signal (204) from the ablation electrode (104); and
      one or more local impedance values (208) between the electrodes; and
   using a machine learning algorithm (212) to classify the tissue which is being ablated, wherein the inputs of the machine learning algorithm (212) are features (210) derived from the ablation-dependent variables, wherein the features correlate with the transmurality of an ablation, and wherein the machine learning algorithm (212) is adapted to output, in real time, a classification of whether the ablation is transmural or non-transmural.
Clause 19. A computer program comprising code means for implementing the method according to claim 18 when said program is run on a processing system.

1. A processing system for determining whether an ablation of tissue of a subject performed using an ablation catheter creates a lesion having a depth that reaches or exceeds a predetermined depth, wherein the ablation catheter comprises at least two electrodes, wherein at least one of the electrodes is an ablation electrode and the other electrode or electrodes are reference electrodes, wherein, during the ablation, electrical signals are measured at the at least two electrodes which electrical signals comprise one or more ablation dependent variables,
the processing system comprising:
an input configured to receive data comprising the one or more ablation dependent variables;
a processor configured to process the received data and:
derive one or more features from the one or more ablation dependent variables, the one or more features correlating with a depth of the lesion resulting from the ablation;
generate an ablation classification result using a machine learning algorithm trained to provide the classification result by processing input data, the classification result comprising an indication whether or not a depth of the lesion resulting from the ablation reaches or exceeds a predetermined depth, wherein the input data of the machine learning algorithm comprises the one or more features and, optionally, also comprises the one or more ablation dependent variables; and
an output configured to provide the classification result.

2. The processing system of claim 1, wherein the ablation dependent variables comprise or consist of:
one or more of the voltages measured at the at least two electrodes,
one or more of the currents measured at the at least two electrodes, and/or
one or more local impedance values between pairs of the at least two electrodes,
preferably wherein the, ablation dependent variables comprise or consist of: an electrical signal from the ablation electrode and one or more local impedance values between pairs of the at least two electrodes;

3. The processing system according to claim 1, wherein the ablation-dependent variables comprise a real component and an imaginary component, and wherein the one or more features are one or more of:
the ratio of the real component of an ablation-dependent variable, at the start of ablation, to the real component of the ablation-dependent variable, at the latest available time during ablation;
the ratio of the imaginary component of an ablation-dependent variable, at the start of ablation to the imaginary component of the ablation-dependent variable, at the latest available time during ablation;
the difference between the real component of an ablation-dependent variable, at the start of ablation, and the real component of the ablation-dependent variable, at the latest available time during ablation; and
the difference between the imaginary component of an ablation-dependent variable, at the start of ablation and the imaginary component of the ablation-dependent variable, at the latest available time during ablation.

4. The processing system according to claim 3, wherein the one or more features are one or more of:
the average value of the ratios of the real components of an ablation-dependent variable over time;
the average value of the ratios of the imaginary components of an ablation-dependent variable over time;
the average value of the differences between the real components of an ablation-dependent variable over time; and
the average value of the differences between the imaginary components of an ablation-dependent variable over time.

5. The processing system according to claim 1, further comprising a memory for storing historic values for the one or more of the ablation-dependent variables, wherein the input data further comprises the historic values.

6. The processing system according to claim 1, wherein the processor is further configured to adapt a decay function to one or more of the ablation-dependent variables over time, and wherein the one or more features comprise or consist of one or more of the coefficients of the decay function for the one or more ablation-dependent variables.

7. The processing system according to claim 1, wherein the one or more features comprise or consist of one or more of:
the average value of the ratios of the absolute value of an ablation-dependent variable, at the start of ablation, to the absolute value of the ablation-dependent variable, at the latest available time during ablation, over time; and
the average value of the differences between the absolute value of an ablation-dependent variable, at the start of ablation, and the absolute value of the ablation-dependent variable, at the latest available time during ablation, over time.

8. The processing system according to claim 1, wherein the processor is further configured to determine exponential coefficients by fitting an exponential function to one or more admittance curves, wherein the admittance curves are based on the one or more local impedance values over time, and wherein the one or more features comprise or consist of the exponential coefficients.

9. The processing system according to claim 1, wherein the processor is further configured to determine the reflected energy and/or power from the tissue, wherein the reflected energy and/or power are calculated based on the electrical signal and the local impedance between the two electrodes of the at least two electrodes that are closest to the tissue (preferably these include at least the ablation electrode), and wherein the one or more features comprise or consist of the reflected energy and/or power.

10. The processing system according to claim 1, wherein the input data of the machine learning algorithm further comprises one or more of:
the duration of ablation;
the ablation generator power; and
the ablation generator electrical current.

11. The processing system according to claim 1, wherein the input data of the machine learning algorithm further comprises the thickness of the tissue.

12. The processing system according to claim 1, wherein the one or more features comprise or consist of the average value of the absolute difference of the phase of an ablation-dependent variable, at the start of ablation, and the phase of the ablation-dependent variable, at the latest available time during ablation, over time.

13. The processing system according to claim 1, wherein the one or more features comprise or consist of the value of the area over a local impedance curve during ablation to a baseline impedance value line, wherein the local impedance curve is based on local impedance values over time.

14. The processing system according to claim 1, wherein the classification result comprises a likelihood indicator indicating how likely it is for the depth of the lesion resulting from the ablation to have reached or exceeded a predetermined depth.

15. The processing system according to claim 1, wherein the classification result further comprises one or both of:
a prediction of the dimensions of the ablation lesion caused by the ablation catheter; and
a prediction of steam pop and/or char occurrence during ablation.

16. A system for determining whether an ablation of tissue of a subject performed using an ablation catheter creates a lesion having a depth that reaches or exceeds a predetermined depth, comprising:
the processing system according to claim 1; and
one or more of:
the ablation catheter and
a user interface having an input for receiving the classification result from the processing system and for providing the classification result to the user of the system, wherein the user interface preferably comprises a display unit for providing a visual indication of the classification result and/or a speaker for providing an audible indication of the classification result and/or a device for providing a tactile indication of the classification result.

17. The system according to claim 16, further comprising a user interface comprising display unit, wherein the display unit is configured to display a map of the tissue being ablated, and the classification result, wherein preferably the classification result is displayed on the tissue.

18. A method for determining whether an ablation of tissue performed by an ablation catheter creates a lesion having a depth that reaches or exceeds a predetermined depth, wherein the ablation catheter comprises at least two electrodes, wherein one of the electrodes is an ablation electrode and the other electrode or electrodes are reference electrodes, wherein during the ablation, electrical signals are measured at the at least two electrodes which electrical signals comprise one or more ablation dependent variables, the method comprising:
receiving data comprising the one or more ablation-dependent variables;
deriving one or more features from the received one or more ablation dependent variables, the one or more features correlating with a depth of the lesion resulting from the ablation;
generating an ablation classification result using a machine learning algorithm trained to provide the classification result by processing input data, the classification result comprising an indication on whether or not a depth of the lesion resulting from the ablation reaches or exceeds the predetermined depth, wherein the input data of the machine learning algorithm comprises the one or more features and, optionally, also comprises the one or more ablation dependent variables; and
optionally, providing the classification result.

19. A computer program comprising instructions for implementing the method of claim 18 when said program is run on a processor.

20. A non-transitory computer readable medium comprising the computer program of claim 19.
